(19) United States
(12) Patent Application Publication
(10) Pub. No.: US 2022/0370155 A1
(43) Pub. Date: Nov. 24, 2022

(54) SURGICAL SYSTEM AND INFORMATION PROCESSING METHOD

(71) Applicant: OLYMPUS CORPORATION, Tokyo (JP)

(72) Inventor: Ryuichi YORIMOTO, Tokyo (JP)

(73) Assignee: OLYMPUS CORPORATION, Tokyo (JP)

(21) Appl. No.: 17/874,690

(22) Filed: Jul. 27, 2022

Related U.S. Application Data
(63) Continuation of application No. PCT/JP2020/008841, filed on Mar. 3, 2020.

(51) Int. Cl. A61B 34/20; A61B 8/12; A61B 8/00; A61B 1/313

(52) U.S. Cl. CPC A61B 34/20; A61B 8/12; A61B 8/463; A61B 1/3132; A61B 2034/2063

(57) ABSTRACT

A surgical system includes: an endoscope capable of acquiring an endoscopic image of a surface of target tissue; an ultrasonic probe capable of acquiring an ultrasonic tomographic image of the target tissue; a treatment instrument; a display; and a controller including a memory and a processor. In response to the ultrasonic probe being inserted into the body cavity and the ultrasonic tomographic image being acquired, the processor is configured to: detect a position of the ultrasonic probe with respect to the endoscope; store the ultrasonic tomographic image associated with the position of the ultrasonic probe; and in a state in which the treatment instrument remains inserted in the body cavity, detect a position of the treatment instrument, read out the stored ultrasonic tomographic image on a basis of the detected position of the treatment instrument, and command the display to display the read-out ultrasonic tomographic image.

This is a continuation of International Application PCT/JP2020/008841, with an international filing date of Mar. 3, 2020, which is hereby incorporated by reference herein in its entirety.

### TECHNICAL FIELD

The present invention relates to a surgical system and a surgical method.

### BACKGROUND ART

There is a known endoscopic surgical system with which treatment is performed by inserting an endoscope and a treatment instrument into the body cavity of a patient through trocars (for example, see PTL 1).

A laparoscope is inserted through a trocar, a treatment instrument is inserted through another trocar, and treatment is performed on tissue in the body cavity.

In such a case, prior to the treatment, an ultrasonic probe is inserted instead of the treatment instrument. Then an ultrasonic tomographic image of a site to be treated is acquired to gain understanding of the inner structure of the tissue. After confirming the inner structure from the ultrasonic tomographic image, a treatment instrument is inserted instead of the ultrasonic probe, and treatment such as tissue excision is performed.

### CITATION LIST

### Patent Literature

{PTL 1} Japanese Unexamined Patent Application, Publication No. H8-275958

### SUMMARY OF INVENTION

An aspect of the present embodment is directed to a surgical system comprising: an endoscope to be inserted into a body cavity and capable of acquiring an endoscopic image of a surface of target tissue; an ultrasonic probe to be inserted into the body cavity and capable of acquiring an ultrasonic tomographic image of the target tissue; a treatment instrument to be inserted into the body cavity; a display capable of displaying the ultrasonic tomographic image acquired by the ultrasonic probe; and a controller that comprises a memory comprising hardware and a processor comprising hardware, the controller being connected to the endoscope, the ultrasonic probe, and the display, wherein, in response to the ultrasonic probe being inserted into the body cavity and the ultrasonic tomographic image being acquired, the processor is configured to: detect a position of the ultrasonic probe with respect to the endoscope; store, in the memory, the ultrasonic tomographic image associated with the position of the ultrasonic probe; and in a state in which the treatment instrument remains inserted in the body cavity, detect a position of the treatment instrument with respect to the endoscope, read out the ultrasonic tomographic image stored in the memory on a basis of the detected position of the treatment instrument, and command the display to display the read-out ultrasonic tomographic image.

Another aspect of the present embodiment is directed to an information processing method comprising: inserting an endoscope and an ultrasonic probe into a body cavity; acquiring a plurality of ultrasonic tomographic images of target tissue by the ultrasonic probe; storing, in a memory comprising hardware, the plurality of ultrasonic tomographic images in association with positions of the ultrasonic probe with respect to the endoscope when these ultrasonic tomographic images are acquired; inserting a treatment instrument into the body cavity instead of the ultrasonic probe; reading out the ultrasonic tomographic image stored in the memory on a basis of a position of the treatment instrument with respect to the endoscpe; and displaying the read-out ultrasonic tomographic image.

Another aspect of the present embodiment is directed to a surgical system comprising: an endoscope that acquires an endoscopic image of a surface of target tissue; a memory comprising hardware, the memory storing a plurality of ultrasonic tomographic images of the target tissue, which has been obtained by the ultrasonic probe inserted into the body cavity, in association with positions of the ultrasonic probe when these ultrasonic tomographic images are acquired, the positions of the ultrasonic probe being detected by processing the endoscopic image; a processor comprising hardware, the processor being configured to: detect a position of a treatment instrument inserted into the body cavity by processing the endoscopic image; and read out, on a basis of the detected position of the treatment instrument, the ultrasonic tomographic image stored in the memory; and a display that displays the ultrasonic tomographic image read out by the processor.

### BRIEF DESCRIPTION OF DRAWINGS

FIG. 1 is a diagram of an example of the application of a surgical system according to one embodiment of the present invention, and illustrates a state in which an ultrasonic probe is inserted into a body cavity.

FIG. 2 is a block diagram of the surgical system illustrated in FIG. 1.

FIG. 3 is a diagram illustrating the position of an ultrasonic scan plane in an endoscopic image acquired in the surgical system illustrated in FIG. 1.

FIG. 4 is a schematic view of a state in which a knife is inserted into the body cavity instead of the ultrasonic probe.

FIG. 5 is a diagram of one example of an image displayed in the surgical system illustrated in FIG. 1 at the stage of performing treatment with a knife.

FIG. 6 is a diagram of one example of an image displayed when the knife is moved from the state illustrated in FIG. 5.

FIG. 7 is a diagram of another example of the image illustrated in FIG. 5.

FIG. 8 is a flowchart of a surgical method according to one embodiment of the present invention.

FIG. 9 is a flowchart of a storing stage illustrated in FIG. 8.

FIG. 10 is a flowchart of a treatment stage illustrated in FIG. 8.

### DESCRIPTION OF EMBODIMENTS

A surgical system 1 and a surgical method according to one embodiment of the present invention will now be described with reference to the drawings.

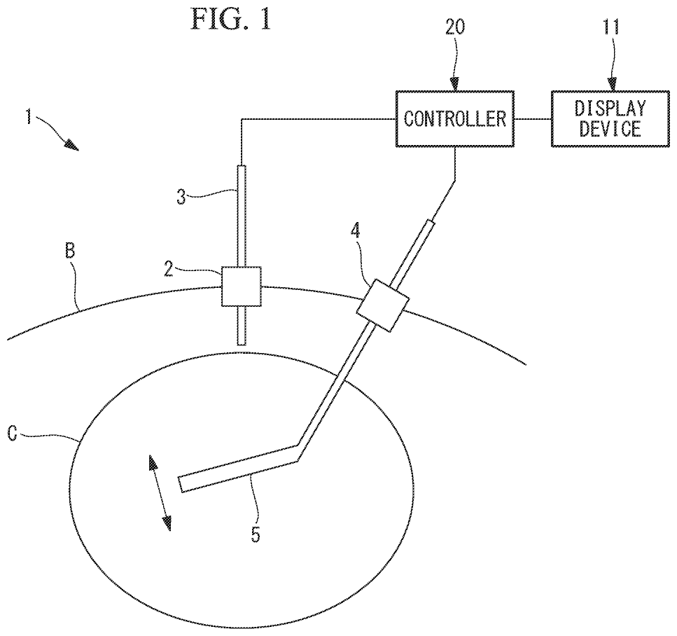

As illustrated in FIG. 1, the surgical system 1 according to this embodiment is applied to a surgery that involves inserting an endoscope 3 into a body cavity through a trocar 2 penetrating through a body wall B of a patient, and performing the surgery while observing the surface of the target tissue C (for example, liver C) with the endoscope 3.

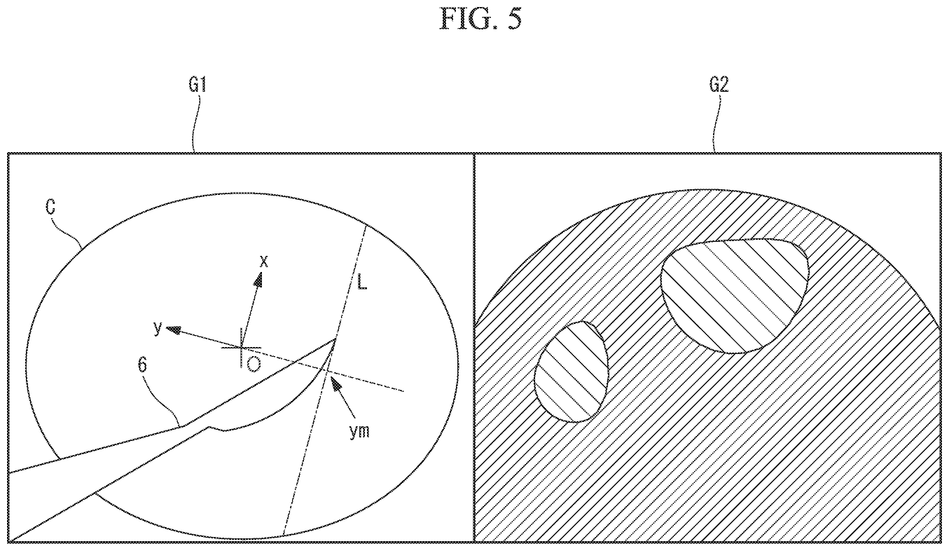

In one embodiment of this surgery, an ultrasonic probe 5 is inserted into the body cavity through another trocar 4. Then an ultrasonic tomographic image (see FIG. 5) G2 of the liver C is acquired while moving the ultrasonic probe 5 over the surface of the liver C in one direction (the arrow direction).

In addition, after acquisition of the ultrasonic tomographic image G2 by the ultrasonic probe 5 is completed, the ultrasonic probe 5 is removed from the trocar 4. Then a treatment instrument 6 (for example, knife 6) for excising an affected site in the liver C is inserted into the body cavity instead of the ultrasonic probe 5. The treatment instrument used may be the knife 6 or any other treatment instrument.

As illustrated in FIG. 1, the surgical system 1 of this embodiment comprises the endoscope 3, a controller 20, and a display (display unit) 11. The endoscope 3 and the ultrasonic probe 5 are connected to the controller 20.

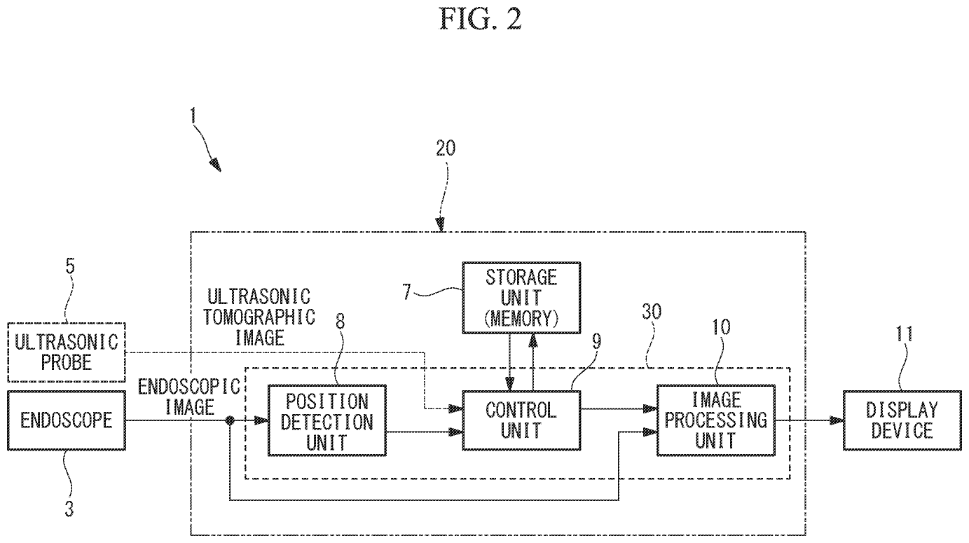

As illustrated in FIG. 2, the controller 20 comprises a storage unit 7, a position detection unit 8, a control unit (including tomographic image read-out unit) 9, and an image processing unit 10.

The storage unit 7 is a storage device, such as a memory. The position detection unit 8, the control unit 9, and the image processing unit 10 are constituted by a processor 30. The display unit 11 is a device to display images, such as a monitor.

The position of the ultrasonic scan plane scanned by the ultrasonic probe 5 when the ultrasonic probe 5 is placed on the surface of the liver C is stored in the storage unit 7. The ultrasonic tomographic image G2 acquired at the same timing is also stored in the storage unit 7 assosiated with the position of the ultrasonic scan plane.

When the ultrasonic tomographic image G2 is stored in the storage unit 7, the position detection unit 8 processes an endoscopic image G1 acquired by the endoscope 3 at a particular frame rate. Thereby, the position of the ultrasonic scan plane is caluculated as the distance to the position of a reference point O in the endoscopic image G1.

When the ultrasonic tomographic image G2 is acquired, as indicated by a broken line in FIG. 2, the ultrasonic probe 5 can be connected to the control unit 9. Then, the control unit 9 stores the position of the ultrasonic scan plane associated with the ultrasonic tomographic image G2 acquired at this timing in the storage unit 7.

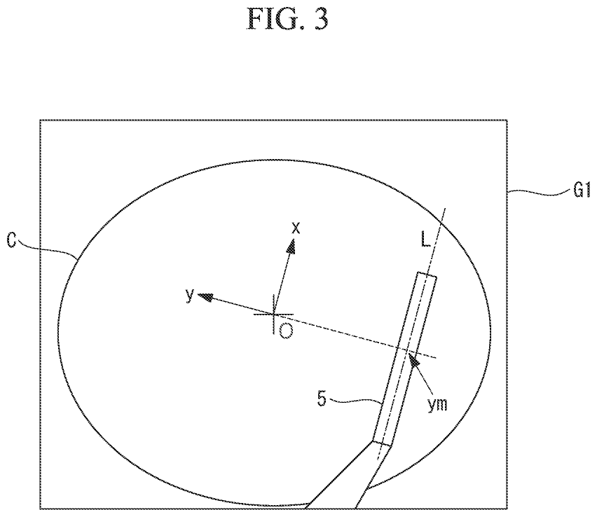

As illustrated in FIG. 3, the reference point O is set at the desired position of the endoscopic image G1. For example, the reference point O may be set at the center position of the endoscopic image G1. Alternatively, one or more feature points within the endoscopic image G1 may be extracted, and any desired position determined with respect to the extracted feature points may be used as the reference point O.

In addition, as illustrated in FIG. 3, for example, when the ultrasonic probe 5 present in the endoscopic image G1 is a bar having a particular width, the ultrasonic scan plane is identified as a straight line L extending in the longitudinal direction at the center position in the width direction. The position of the scan plane may be determined by calculating the distance from the reference point O to the straight line L.

As illustrated in FIG. 3, by using the set reference point O as the origin, the control unit 9 can set an x axis that extends in a direction parallel to the straight line L. Also, the control unit 9 can set a y axis that extends in a direction orthogonal to the x axis.

The ultrasonic tomographic image G2 is acquired by the ultrasonic probe 5 at a particular frame rate while the ultrasonic probe 5 is moved in the width direction, that is, as indicated in the arrow in FIG. 1, while the ultrasonic probe 5 is moved in a direction intersecting the ultrasonic scan plane. Each time an ultrasonic tomographic image G2 is acquired, the distance yn (y coordinate) from the reference point O serving as the origin to the scan plane in the y-axis direction is calculated, and the distance yn associated with the ultrasonic tomographic image G2 is stored in the storage unit 7.

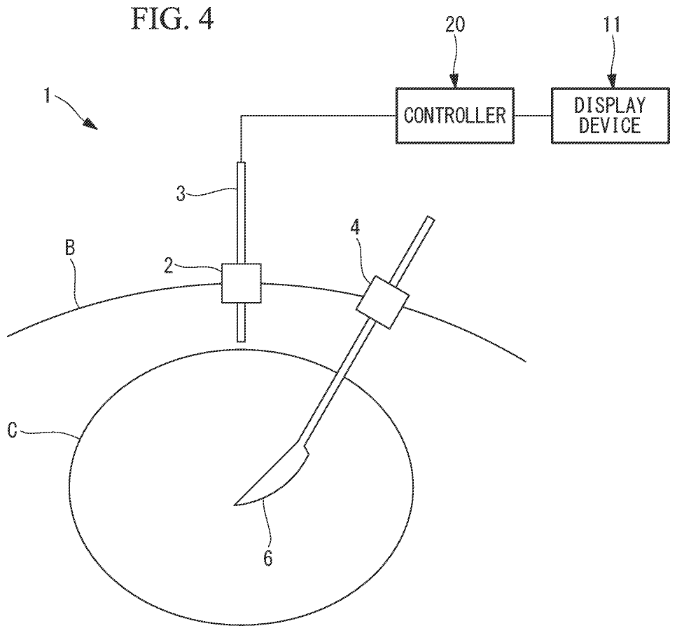

As illustrated in FIG. 4, at the stage where the knife 6 instead of the ultrasonic probe 5 is inserted into the body cavity to treat the liver C, the position detection unit 8 processes the endoscopic image G1 and detects the position of the knife 6 inserted into the body cavity. Specifically, in the endoscopic image G1 acquired by the endoscope 3, the distal end position of the knife 6 in the endoscopic image G1 is extracted. Next, as illustrated in FIG. 5, in the coordinate system set in FIG. 3, the y coordinate ym of the extracted distal end position of the knife 6 is detected as the position of the knife 6.

Next, the position of the knife 6 detected by the position detection unit 8 and the endoscopic image G1 currently acquired by the endoscope 3 are input to the control unit 9. The control unit 9 reads out from the storage unit 7 the ultrasonic tomographic image G2 stored in association with the input position of the knife 6. Specifically, the ultrasonic tomographic image G2 acquired by the ultrasonic probe 5 at the same position as the position of the knife 6 detected by the position detection unit 8 is read out.

Furthermore, as illustrated in FIG. 5, the control unit 9 sends the read-out ultrasonic tomographic image G2 to the image processing unit 10. The image processing unit 10 generates a composite image in which the ultrasonic tomographic image G2 sent from the control unit 9 and the endoscopic image G1 of the current surface of the liver C input from the endoscope 3 are arranged side-by-side, and sends the composite image to the display unit 11. The display unit 11 displays the composite image sent from the image processing unit 10. A surgical method according to this embodiment having such features will now be described.

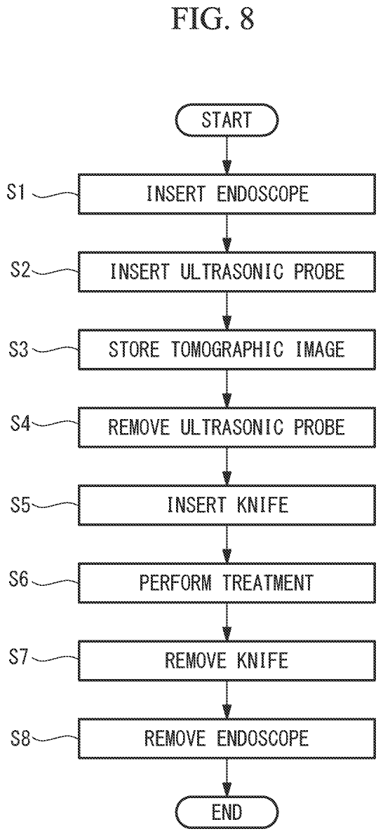

As illustrated in FIG. 8, in the surgical method according to this embodiment, the endoscope 3 is inserted into the body cavity through the trocar 2 (step S1), and the ultrasonic probe 5 is inserted into the body cavity through the trocar 4 (step S2).

Next, by using the endoscopic image G1 acquired by the endoscope 3 and the ultrasonic tomographic image G2 acquired by the ultrasonic probe 5, the position information of the ultrasonic probe 5 in the screen detected by the position detection unit 8 is associated with the ultrasonic tomographic image G2 and is stored in the storage unit 7 (tomographic image storing step S3), and the ultrasonic probe 5 is removed from the body cavity (step S4).

Next, in order to dissect the affected site of the liver C, the knife 6 is inserted into the body cavity through the trocar 4 (step S5), the endoscopic image G1 associated with the ultrasonic tomographic image G2 is displayed on the display 11 (treatment step S6), and the liver C is treated. Upon completion of the treatment, the knife 6 is removed from the body cavity (step S7), and the endoscope 3 is removed (step S8), whereupon the procedure ends.

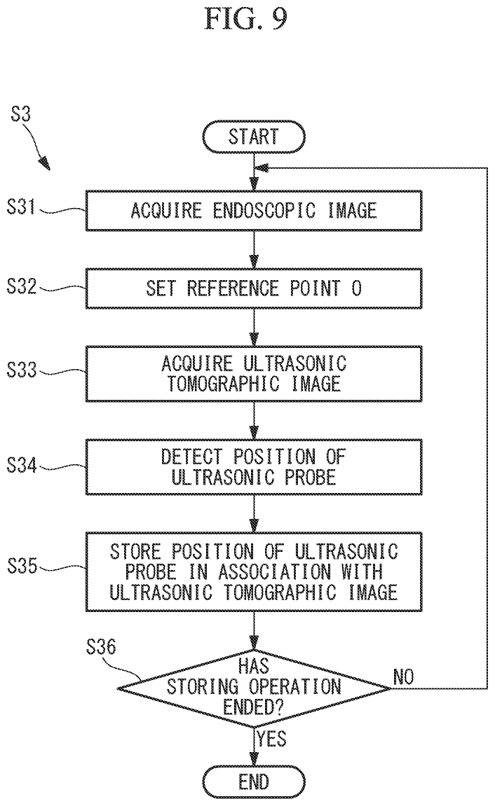

More specifically, as illustrated in FIG. 9, in the tomographic image storing step S3, an endoscopic image G1 is acquired by the endoscope 3 (step S31), and a reference point O is set at the center position of the acquired endoscopic image G1 (step S32).

Next, an ultrasonic tomographic image G2 of the liver C is acquired by the ultrasonic probe 5 (step S33), and, each time an ultrasonic tomographic image G2 is acquired, the position detection unit 8 calculates the position information of the ultrasonic probe 5 on the screen with respect to the reference point O, in other words, the distance yn from the reference point O to the scan plane in the y-axis direction (step S34).

Subsequently, the distance yn from the reference point O in the xy coordinate system set by the control unit 9 in the endoscopic image G1 is associated with the ultrasonic tomographic image G2 and is stored in the storage unit 7 (step S35), whether the storing operation is finished or not is determined (step S36), and, if the storing operation is to be continued, the steps from the step S31 are repeated, and if the storing operation is to be ended, step S4 is executed.

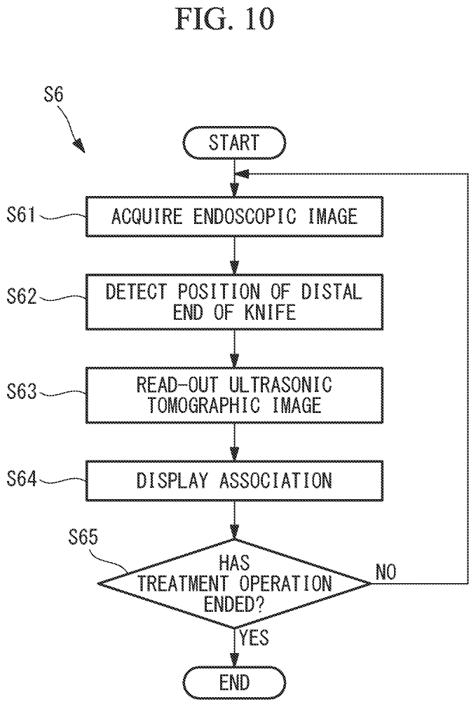

Furthermore, as illustrated in FIG. 10, in the treatment step S6, an endoscopic image G1 is acquired by the endoscope 3 (step S61), and the acquired endoscopic image G1 is processed by the position detection unit 8 to detect the distal end position of the knife 6 (step S62). Next, the control unit 9 reads out the ultrasonic tomographic image G2 associated with the distal end position of the knife 6 from the storing unit 7 (step S63), and the image processing unit 10 displays the endoscopic image G1 and the associated ultrasonic tomographic image G2 on the display unit 11 (step S64). Subsequently, whether the treatment operation is finished or not is determined (step S65);, if the treatment operation is to be continued, the steps from the step S61 are repeated, and if the treatment operation is to be ended, step S7 is executed.

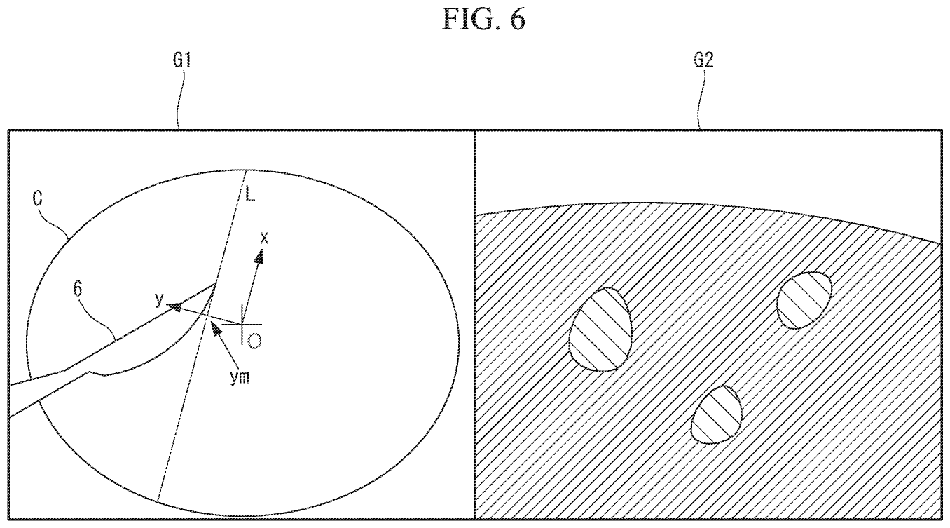

In this manner, as long as the position of the knife 6 is detected at a particular sampling period by the position detection unit 8, as illustrated in FIGS. 5 and 6, an ultrasonic tomographic image G2 taken along a scan plane that passes through the distal end of the knife 6 and updated each time the knife 6 is moved on the endoscopic image G1 is displayed on the display unit 11. This provides an advantage in that, even when the ultrasonic probe 5 is not used during dissection using the knife 6, the accurate inner structure of the site to be dissected can be quickly confirmed. Thus, it is not necessary to alternate between the observation and the treatment by removing and inserting the ultrasonic probe 5 and the knife 6, and the liver C can be easily and accurately dissected with less trouble.

Although the image processing unit 10 generates a composite image in which the endoscopic image G1 and the ultrasonic tomographic image G2 are arranged side-by-side in this embodiment, alternatively, the endoscopic image G1 and the ultrasonic tomographic image G2 may be displayed in separate screens instead.

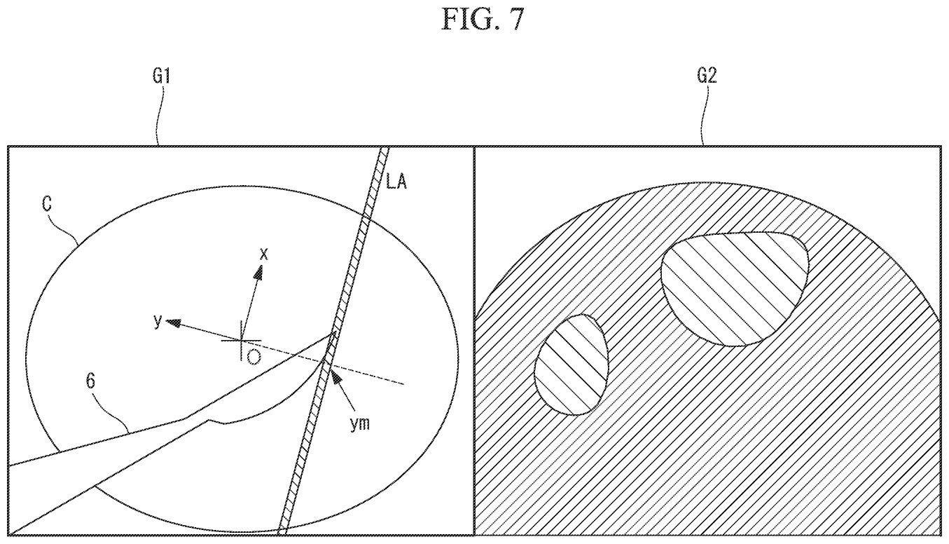

In this embodiment, the image processing unit 10 may receive the information regarding the position of the ultrasonic tomographic image G2 from the control unit 9, and, as illustrated in FIG. 7, may generate a composite image obtained by superimposing a straight line LA (indicator sign) that indicates the ultrasonic scan plane onto the endoscopic image G1. This provides an advantage in that the operator can clearly visually identify, through the straight line LA, in which direction the ultrasonic tomographic image G2 displayed next to the endoscopic image G1 extends with respect to the liver C displayed in the endoscopic image G1. In other words, when an affected site is found in the ultrasonic tomographic image G2, the affected site can be more accurately reached by dissecting along the straight line LA.

Although the control unit 9 sets the rectangular coordinate system xy with respect to the reference point O at the center position of the endoscopic image G1 in this embodiment, alternatively, the y coordinate may be set in any direction that intersects the x direction. In this manner also, the ultrasonic tomographic image G2 can be unambiguously associated with the distal end position of the knife 6.

Even when the endoscope 3 is moved during the treatment, by setting the reference point O on the basis of the feature points in the endoscopic image G1, the reference points 0 in the endoscopic images G1 obtained before and after the movement can be made coincident.

Alternatively, the reference points 0 in the endoscopic images G1 obtained before and after the movement of the endoscope 3 may be made coincident by detecting the amount of movement of the endoscope 3.

In this manner, even when the visual field of the endoscope 3 during acquisition of the ultrasonic tomographic image by the ultrasonic probe 5 is different from the visual field of the endoscope 3 during the treatment using the knife 6, an ultrasonic tomographic image associated with the position of the knife 6 can be read out.

The ultrasonic probe 5 and the knife 6 serving as a treatment instrument may be inserted and removed through the same trocar 4 or may be inserted simultaneously using separate trocars 4. In this case also, there is an advantage in that acquisition of the ultrasonic tomographic image G2 by the ultrasonic probe 5 and the treatment using the knife 6 serving as the treatment instrument do not have to alternate.

As a result, the above-described embodiment also leads to the following aspects.

An aspect of the present embodiment is directed to a surgical system comprising: an endoscope to be inserted into a body cavity and capable of acquiring an endoscopic image of a surface of target tissue; an ultrasonic probe to be inserted into the body cavity and capable of acquiring an ultrasonic tomographic image of the target tissue; a treatment instrument to be inserted into the body cavity; a display capable of displaying the ultrasonic tomographic image acquired by the ultrasonic probe; and a controller that comprises a memory and a processor, the controller being connected to the endoscope, the ultrasonic probe, and the display, in which, in response to the ultrasonic probe being inserted into the body cavity and the ultrasonic tomographic image being acquired, the processor is configured to: detect a position of the ultrasonic probe on a basis of the endoscopic image acquired by the endoscope; store, in the memory, the ultrasonic tomographic image associated with the position of the ultrasonic probe; and in a state in which the treatment instrument remains inserted in the body cavity, detect a position of the treatment instrument on the basis of the endoscopic image acquired by the endoscope, read out the ultrasonic tomographic image stored in the memory on the basis of the detected position of the treatment instrument, and command the display to display the read-out ultrasonic tomographic image.

Another aspect of the present embodiment is directed to a surgical method comprising: storing a tomographic image; and treating, in which the storing of the tomographic image comprises inserting an endoscope and an ultrasonic probe into a body cavity, acquiring a plurality of ultrasonic tomographic images of target tissue by the ultrasonic probe while acquiring an endoscopic image of a surface of the target tissue, and storing, in a storage, the acquired ultrasonic tomographic images in association with positions of the ultrasonic probe when these ultrasonic tomographic images are acquired, the positions of the ultrasonic probe being detected by processing the endoscopic image, and the treating comprises inserting a treatment instrument into the body cavity instead of the ultrasonic probe, detecting a position of the treatment instrument by processing the endoscopic image while acquiring the endoscopic image of the surface of the target tissue, reading out the ultrasonic tomographic image stored in the storage unit on a basis of the detected position of the treatment instrument, and displaying the read-out ultrasonic tomographic image.

Another aspect of the present embodiment is directed to a surgical system comprising: an endoscope that acquires an endoscopic image of a surface of target tissue; a storage that stores a plurality of ultrasonic tomographic images of the target tissue, which has been obtained by the ultrasonic probe inserted into the body cavity, in association with positions of the ultrasonic probe when these ultrasonic tomographic images are acquired, the positions of the ultrasonic probe being detected by processing the endoscopic image; a position detection unit that detects a position of a treatment instrument inserted into the body cavity by processing the endoscopic image; a tomographic image read-out unit that reads out, on the basis of the position of the treatment instrument detected by the position detection unit, the ultrasonic tomographic image stored in the storage; and a display that displays the ultrasonic tomographic image read out by the tomographic image read-out unit.

According to this aspect, ultrasonic tomographic images of the target tissue acquired by inserting the ultrasonic probe into the body cavity are associated with the positions of the ultrasonic probe when the ultrasonic tomographic images are obtained, the positions being detected by processing the endoscopic image, and are stored in the storage unit. Then, when the target tissue is treated with the treatment instrument inserted into the body cavity, the position detection unit detects the position of the treatment instrument detected by processing the endoscopic image acquired by the endoscope.

Next, on the basis of the detected position of the treatment instrument, the ultrasonic tomographic image stored in the storage is read out by the tomographic image read-out unit, and displayed on the display. In this manner, when the treatment instrument is moved in the body cavity, the ultrasonic tomographic image of the target tissue at the position associated with the position of the treatment instrument is displayed on the display. As a result, the operator can confirm the accurate inner structure of the site to be treated without having to use the ultrasonic probe during performing the treatment with the treatment instrument.

In the aspect described above, the position of the ultrasonic probe may be a position in a direction that intersects an ultrasonic scan plane scanned by the ultrasonic probe.

According to this feature, the ultrasonic tomographic image can be stored and be easily read out. In other words, when the ultrasonic probe is placed on the surface of the target tissue and actuated, an ultrasonic tomographic image that extends along the ultrasonic scan plane is acquired. As the ultrasonic probe is translationally moved in one direction intersecting the scan plane, multiple ultrasonic tomographic images aligning in the translational movement direction can be acquired. Thus, the position of the ultrasonic tomographic image can be easily identified by merely storing the position of the ultrasonic probe in the direction intersecting the ultrasonic scan plane in association with the ultrasonic tomographic image.

In the aspect described above, the position of the treatment instrument may be a position of a distal end of the treatment instrument.

When the treatment is performed by bringing the treatment instrument close to the target tissue while endoscopically observing the surface of the target tissue in the body cavity, the operator focuses most on the position of the distal end of the treatment instrument; thus, it is most convenient if the ultrasonic tomographic image at that position is read out.

According to this feature, since the ultrasonic tomographic image at the position of the distal end of the treatment instrument is displayed as the treatment instrument is moved on the surface of the target tissue, the inner structure of the site to be treated can be more accurately confirmed.

In the aspect described above, the position detection unit may set a reference point in the endoscopic image and detect the position of the ultrasonic probe and the position of the treatment instrument by calculating distances from the reference point.

According to this feature, the position of the treatment instrument can be easily detected from the endoscopic image used during the treatment of the target tissue.

In the aspect described above, the display unit may display the endoscopic image and the ultrasonic tomographic image, and display an indicator sign that indicates the position of the ultrasonic tomographic image and that is superimposed onto the endoscopic image.

According to this feature, the indicator sign superimposed on the endoscopic image allows the operator to instantly identify the position of the target tissue that the displayed ultrasonic tomographic image shows the inner structure for.

In the aspect described above, the indicator sign may be a straight line that extends along an ultrasonic scan plane.

According to this feature, the position of the ultrasonic tomographic image can be easily and accurately displayed on the endoscopic image.

The present embodiment offers an advantage in that the accurate inner structure of a site to be treated can be efficiently confirmed without removing and inserting the ultrasonic probe and the treatment instrument.

### REFERENCE SIGNS LIST

1 surgical system

3 endoscope

5 ultrasonic probe

6 knife (treatment instrument)

7 storage unit

8 position detection unit

9 control unit (tomographic image read-out unit)

11 display (display unit)

30 processor

C liver (target tissue)

G1 endoscopic image

G2 ultrasonic tomographic image

O reference point

S3 tomographic image storing step

S6 treatment step

1. A surgical system comprising:
an endoscope to be inserted into a body cavity and capable of acquiring an endoscopic image of a surface of target tissue;
an ultrasonic probe to be inserted into the body cavity and capable of acquiring an ultrasonic tomographic image of the target tissue;
a treatment instrument to be inserted into the body cavity;
a display capable of displaying the ultrasonic tomographic image acquired by the ultrasonic probe; and
a controller that comprises a memory and a processor, the controller being connected to the endoscope, the ultrasonic probe, and the display,
wherein:
in response to the ultrasonic probe being inserted into the body cavity and the ultrasonic tomographic image being acquired,
the processor is configured to:
detect a position of the ultrasonic probe with respect to the endoscope;
store the ultrasonic tomographic image associated with the position of the ultrasonic probe in the memory; and
in a state in which the treatment instrument remains inserted in the body cavity, detect a position of the treatment instrument with respect to the endoscope, read out the ultrasonic tomographic image stored in the memory on a basis of the detected position of the treatment instrument, and command the display to display the read-out ultrasonic tomographic image.

2. The surgical system according to claim 1,
wherein, in response to the ultrasonic probe being inserted into the body cavity and the ultrasonic tomographic image being acquired,
the processor is configured to detect, on the basis of the endoscopic image acquired by the endoscope, a position of the ultrasonic probe in a direction intersecting an ultrasonic scan plane.

3. The surgical system according to claim 2, wherein the processor is configured to:
set a reference point on the endoscopic image, and
detect the position of the ultrasonic probe from a distance between the reference point and the ultrasonic scan plane.

4. The surgical system according to claim 1, wherein, in the state in which the treatment instrument remains inserted in the body cavity, the processor is configured to detect a position of a distal end of the treatment instrument on the basis of the endoscopic image acquired by the endoscope.

5. The surgical system according to claim 4, wherein the processor is configured to:
set a reference point on the endoscopic image, and
detect a position of the treatment instrument from a distance between the reference point and the distal end of the treatment instrument.

6. The surgical system according to claim 1, wherein the processor is configured to set a reference point on the endoscopic image and calculate a distance between the reference point and the ultrasonic probe and a distance between the reference point and the treatment instrument so as to detect positions of the ultrasonic probe and the treatment instrument.

7. The surgical system according to claim 6, wherein the processor is configured to command the display to display the endoscopic image and the ultrasonic tomographic image and to superimpose and display an indicator sign on the endoscopic image, the indicator sign indicating the position of the ultrasonic probe when the ultrasonic tomographic image displayed on the display is acquired.

8. The surgical system according to claim 7, wherein the processor is configured to command the display to display, as the indicator sign, a straight line that extends along an ultrasonic scan plane.

9. The surgical system according to claim 1, wherein the processor is configured to read out, from the memory, the ultrasonic tomographic image acquired by the ultrasonic probe at a same position as the detected position of the treatment instrument.

10. An information processing method comprising:
inserting an endoscope and an ultrasonic probe into a body cavity;
acquiring a plurality of ultrasonic tomographic images of target tissue by the ultrasonic probe;
storing, in a memory comprising hardware, the plurality of ultrasonic tomographic images in association with positions of the ultrasonic probe with respect to the endoscope when these ultrasonic tomographic images are acquired;
inserting a treatment instrument into the body cavity instead of the ultrasonic probe;
reading out the ultrasonic tomographic image stored in the memory on a basis of a position of the treatment instrument with respect to the endoscpe; and
displaying the read-out ultrasonic tomographic image.

11. The information processing method according to claim 10, further comprising:
detecting, on a basis of an endoscopic image acquired by the endoscope, a position of the ultrasonic probe in a direction intersecting an ultrasonic scan plane to acquire the position of the ultrasonic probe.

12. The information processing method according to claim 11, further comprising:
setting a reference point on the endoscopic image and detecting the position of the ultrasonic probe from a distance between the reference point and the ultrasonic scan plane to acquire the position of the ultrasonic probe.

13. The information processing method according to claim 11, further comprising:
setting a reference point on the endoscopic image and acquiring, on the basis of the endoscopic image acquired by the endoscope, a distance between the reference point and the treatment instrument as the position of the treatment instrument.

14. The information processing method according to claim 11, further comprising:
setting a reference point on the endoscopic image and calculating a distance between the reference point and the ultrasonic probe and a distance between the reference point and the treatment instrument so as to detect positions of the ultrasonic probe and the treatment instrument.

15. A surgical system comprising:
an endoscope that acquires an endoscopic image of a surface of target tissue;
a memory comprising hardware, the memory storing a plurality of ultrasonic tomographic images of the target tissue, which has been obtained by the ultrasonic probe inserted into the body cavity, in association with positions of the ultrasonic probe when these ultrasonic tomographic images are acquired, the positions of the ultrasonic probe being detected by processing the endoscopic image;
a processor comprising hardware, the processor being configured to:
detect a position of a treatment instrument inserted into the body cavity by processing the endoscopic image; and
read out, on a basis of the detected position of the treatment instrument, the ultrasonic tomographic image stored in the memory; and
a display that displays the ultrasonic tomographic image read out by the processor.

16. The surgical system according to claim 15, wherein the position of the ultrasonic probe is a position in a direction intersecting an ultrasonic scan plane scanned by the ultrasonic probe.

17. The surgical system according to claim 15, wherein the position of the treatment instrument is a position of a distal end of the treatment instrument.

18. The surgical system according to claim 15, wherein the detecting of the position of the treatment instrument sets a reference point in the endoscopic image and detects the position of the ultrasonic probe and the position of the treatment instrument by calculating distances from the reference point.

19. The surgical system according to claim 18, wherein the display is configured to display the endoscopic image and the ultrasonic tomographic image, and superimpose and display an indicator sign onto the endoscopic image, the indicator sign indicating the position of the ultrasonic tomographic image.

20. The surgical system according to claim 19, wherein the indicator sign is a straight line that extends along an ultrasonic scan plane.
